(19) United States
(12) Patent Application Publication
(10) Pub. No.: US 2025/0118391 A1
(43) Pub. Date: Apr. 10, 2025

(54) DEEP-LEARNING BASED METHODS FOR VIRTUAL SCREENING OF MOLECULES FOR MICRO RIBONUCLEIC ACID (MIRNA) DRUG DISCOVERY

(71) Applicants: UNIVERSITY OF CENTRAL FLORIDA RESEARCH FOUNDATION, INC., Orlando, FL (US); THE REGENTS OF THE UNIVERSITY OF CALIFORNIA, Oakland, CA (US)

(72) Inventors: Jiann-Shiun YUAN, Orlando, FL (US); Arash KESHAVARZI ARSHADI, Orlando, FL (US); Milad SALEM, Orlando, FL (US); Hani GOODARZI, San Francisco, CA (US)

(21) Appl. No.: 18/836,064

(22) PCT Filed: Jan. 10, 2023
(86) PCT No.: PCT/US2023/060385

§ 371 Date: Aug. 6, 2024

(51) Int. Cl. G16B 40/00; C40B 30/06; G16B 35/20

(52) U.S. Cl. CPC G16B 40/00; C40B 30/06; G16B 35/20

(57) ABSTRACT

A method for training a deep learning model configured for virtual screening of molecules for micro ribonucleic acid (miRNA) drug discovery includes creating a training dataset including a plurality of assay datasets. The method also includes training the deep learning model to learn a plurality of tasks; performing, using the deep learning model, inference for the respective task associated with at least one miRNA assay dataset; and evaluating respective performance metrics associated with the respective predictions for each of the plurality of tasks. The method further includes selecting a set of the plurality of assay datasets for training; and training the deep learning model to learn a set of the plurality of tasks using the set of the plurality of assay datasets. The trained deep learning model is configured to predict molecules capable of affecting a target miRNA.

### CROSS-REFERENCE TO RELATED APPLICATIONS

This application claims the benefit of U.S. provisional patent application No. 63/309,132, filed on Feb. 11, 2022, and titled “DEEP-LEARNING BASED METHODS FOR VIRTUAL SCREENING OF MOLECULES FOR MICRO RIBONUCLEIC ACID (miRNA) DRUG DISCOVERY,” the disclosure of which is expressly incorporated herein by reference in its entirety.

### BACKGROUND

As a class of short non-coding ribonucleic acids (RNAs), Micro RNAs (sometimes referred to herein as “microRNAs” or “miRNAs”) are among the essential regulators of cell homeostasis. Micro RNAs play a significant role in the regulation of genes, therefore, deserve attention and need to be studied. One field that is influenced heavily by different types of miRNAs is oncology. For example, miRNAs oversee gene expression and suppress protein synthesis which affects cellular transcription and translation on many levels. The dysregulation of these proteins has been shown to contribute to cancer progression, which is the second leading cause of death worldwide. The genes coding for MicroRNAs driving cancer would function both as oncogenes and tumor suppressor genes.

Because microRNA can regulate a variety of target proteins, they play an important role in the development of various oncogenic hallmarks, such as metastasis, angiogenesis, and resistance to apoptosis. In fact, there is evidence that they may contribute to the suppression of the immune response within the tumor microenvironment. It has been established that microRNAs are related to drug resistance in many studies. According to these studies, microRNAs may contribute to the development of resistance in tumors, one of the leading causes of cancer-related death in the world. Among cancers that are resistant to chemotherapy, miR-21 dysregulation has been found in ovary, gallbladder, colorectal, and pancreatic cancers.

Via targeting the right miRNAs, scientists can control their effects and manipulate gene expression. Their basic functions are mostly silencing messenger RNAs (mRNAs) which are the main builders of different proteins. Targeting and inhibiting miRNA can be used to battle diseases such as cancer and Alzheimer [A. Esquela-Kerscher and F. J. Slack, “Oncomirs-microRNAs with a role in cancer,” Nature Reviews Cancer, vol. 6, no. 4, pp. 259-269, 2006 Apr. 1 2006, doi: 10.1038/nrc1840]. One suitable tool for targeting miRNAs are small molecules, and if designed correctly, they can permeate inside the cell and interact with miRNAs.

It has been difficult, however, to structurally inhibit the activity of RNAs, especially miRNAs, with small molecules. Typically, miRNAs have a very small dynamic structure and thus differ from proteins in terms of functionality. Since microRNAs lack the usual pockets found in proteins, or the pockets are shallower, conventional docking approaches have not been suitable for microRNAs. Furthermore, structurally binding a small molecule to microRNAs does not necessarily impair their functionality. Through inhibition of microRNA upstream modulators, it would be possible to inhibit their activity, but these modulators are largely unknown, and therefore difficult to target. As a whole, microRNAs are still considered largely undruggable.

It would therefore be desirable to provide a platform that can predict the interaction between a given small molecule and a targeted miRNA (e.g., molecules capable of disrupting the miRNA activities through inhibiting the miRNA itself, or any protein upstream important for the miRNA). One of the main challenges of this task is that RNA does not have a rigid and well-known structure like proteins, therefore targeting them by using structure-based drug discovery (SBDD) has not been successful. In the last decades people have tried to use the protein based SBDD models on RNAs, but they are not effective or successful due to the dynamic nature of the RNA structure. Even if it is assumed that the protein based SBDD models work perfectly on RNAs, there has not been enough identified RNA structures to work on. Therefore, SBDD is not the right approach for RNA drug discovery. Although there have been some advances in the prediction of the RNA structures using Deep Learning lately, they are based on small training samples and limited applications. Thus, SBDD is not practical in this time [R. J. L. Townshend et al., “Geometric deep learning of RNA structure,” Science, vol. 373, no. 6558, pp. 1047-1051, 2021, doi: doi: 10.1126/science.abe5650]. The situation is even worse for smaller RNAs like miRNAs since they are more dynamic structures.

### SUMMARY

A method for training a deep learning model configured for virtual screening of molecules for micro ribonucleic acid (miRNA) drug discovery is described herein. The method includes creating a training dataset including a plurality of assay datasets, where the plurality of assay datasets include at least one miRNA assay dataset. The method also includes training the deep learning model to learn a plurality of tasks using the training dataset, where each of the plurality of assay datasets is associated with a respective task; performing, using the deep learning model, inference for the respective task associated with the at least one miRNA assay dataset, where the deep learning model outputs a respective prediction for each of the plurality of tasks; and evaluating respective performance metrics associated with the respective predictions for each of the plurality of tasks. The method further includes selecting a set of the plurality of assay datasets for training based on the respective performance metrics; and training the deep learning model to learn a set of the plurality of tasks using the set of the plurality of assay datasets. The set of the plurality of assay datasets includes the at least one miRNA assay dataset, and the trained deep learning model is configured to predict molecules capable of affecting a target miRNA.

Additionally, in some implementations, the step of training the deep learning model to learn the plurality of tasks using the training dataset is performed in a multi-task manner.

Alternatively or additionally, the step of evaluating respective performance metrics associated with the respective predictions for each of the plurality of tasks includes calculating respective scores based on a comparison of the respective predictions for each of the plurality of tasks to ground truth labels for the respective task associated with the at least one miRNA assay dataset. Optionally, the set of the plurality of assay datasets are associated with respective scores greater than a threshold.

Alternatively or additionally, the target miRNA is miR-21.

Alternatively or additionally, the trained deep learning model is configured to predict molecules that disrupt activities of the target miRNA.

Alternatively or additionally, the trained deep learning model is configured to predict molecules that inhibit activity of the target miRNA or a protein upstream of the target miRNA.

Alternatively or additionally, the deep learning model is a graph convolutional neural network (GCNN).

Alternatively or additionally, the training dataset includes a plurality of molecules expressed in a computer-readable format. Optionally, the computer-readable format is simplified molecular input line entry system (SMILES) notation.

A method for screening of molecules for micro ribonucleic acid (miRNA) drug discovery is also described herein. The method includes providing a deep learning model trained according to the techniques described herein. The method also includes inputting an inference dataset into the trained deep learning model; and predicting, using the trained deep learning model, a plurality of molecules capable of interacting with the target miRNA. The method further includes performing in vitro testing on at least one of the plurality of molecules predicted as capable of affecting the target miRNA.

Additionally, in some implementations, the method further includes providing respective uncertainty scores for each of the plurality of molecules predicted as capable of interacting with the target miRNA; and selecting the at least one of the plurality of molecules on which in vitro testing is performed based, at least in part, on the respective uncertainty scores.

Alternatively or additionally, in some implementations, the method further includes clustering the plurality of molecules predicted as capable of interacting with the target miRNA into a plurality of clusters; and selecting the at least one of the plurality of molecules on which in vitro testing is performed based, at least in part, on at least two of the plurality of clusters.

Alternatively or additionally, in some implementations, the method further includes inputting the plurality of molecules predicted as capable of interacting with the target miRNA into a first machine learning model; predicting, using the first machine learning model, respective toxicity metrics for the plurality of molecules predicted as capable of interacting with the target miRNA; and selecting the at least one of the plurality of molecules on which in vitro testing is performed based, at least in part, on the respective toxicity metrics.

Alternatively or additionally, in some implementations, the method further includes predicting, using the second machine learning model, respective dicer activity metrics for the plurality of molecules predicted as capable of interacting with the target miRNA; and selecting the at least one of the plurality of molecules on which in vitro testing is performed based, at least in part, on the respective dicer activity metrics.

Alternatively or additionally, in some implementations, the inference dataset includes a plurality of molecules expressed in a computer-readable format. Optionally, the computer-readable format is simplified molecular input line entry system (SMILES) notation.

A method for virtual screening of molecules for micro ribonucleic acid (miRNA) drug discovery is also described herein. The method includes providing a trained deep learning model; inputting a molecule into the trained deep learning model, wherein the molecule is expressed in a computer-readable format; and predicting, using the trained deep learning model, that the molecule is capable of interacting with a target miRNA.

Additionally, structural information about the target miRNA is not a feature input into the trained deep learning model.

Alternatively or additionally, the trained deep learning model is a graph convolutional neural network (GCNN).

An example system for training a deep learning model configured for virtual screening of molecules for micro ribonucleic acid (miRNA) drug discovery is also described herein. The system includes at least one processor, and at least one memory operably coupled to the at least one processor. The at least one memory has computer-executable instructions stored thereon that, when executed by the at least one processor, cause the at least one processor to: receive a training dataset including a plurality of assay datasets, where the plurality of assay datasets includes at least one miRNA assay dataset; and train the deep learning model to learn a plurality of tasks using the training dataset, where each of the plurality of assay datasets is associated with a respective task. The at least one memory has further computer-executable instructions stored thereon that, when executed by the at least one processor, cause the at least one processor to: perform, using the deep learning model, inference for the respective task associated with the at least one miRNA assay dataset, where the deep learning model outputs a respective prediction for each of the plurality of tasks; evaluate respective performance metrics associated with the respective predictions for each of the plurality of tasks; and select a set of the plurality of assay datasets for training based on the respective performance metrics. The at least one memory has further computer-executable instructions stored thereon that, when executed by the at least one processor, cause the at least one processor to: train the deep learning model to learn a set of the plurality of tasks using the set of the plurality of assay datasets, where the set of the plurality of assay datasets includes the at least one miRNA assay dataset, and where the trained deep learning model is configured to predict molecules capable of affecting a target miRNA.

Additionally, in some implementations, the step of training the deep learning model to learn the plurality of tasks using the training dataset is performed in a multi-task manner.

Alternatively or additionally, the step of evaluating respective performance metrics associated with the respective predictions for each of the plurality of tasks includes calculating respective scores based on a comparison of the respective predictions for each of the plurality of tasks to ground truth labels for the respective task associated with the at least one miRNA assay dataset. Optionally, the set of the plurality of assay datasets are associated with respective scores greater than a threshold.

Alternatively or additionally, the target miRNA is miR-21.

Alternatively or additionally, the trained deep learning model is configured to predict molecules that disrupt activities of the target miRNA.

Alternatively or additionally, the trained deep learning model is configured to predict molecules that inhibit activity of the target miRNA or a protein upstream of the target miRNA.

Alternatively or additionally, the deep learning model is a graph convolutional neural network (GCNN).

Alternatively or additionally, the training dataset includes a plurality of molecules expressed in a computer-readable format. Optionally, the computer-readable format is simplified molecular input line entry system (SMILES) notation.

An example system for virtual screening of molecules for micro ribonucleic acid (miRNA) drug discovery is also described herein. The system includes a trained deep learning model, at least one processor, and at least one memory operably coupled to the at least one processor. The at least one memory has computer-executable instructions stored thereon that, when executed by the at least one processor, cause the at least one processor to: input a molecule into the trained deep learning model, where the molecule is expressed in a computer-readable format; and predict, using the trained deep learning model, that the molecule is capable of interacting with a target miRNA.

Additionally, structural information about the target miRNA is not a feature input into the trained deep learning model.

Alternatively or additionally, the trained deep learning model is a graph convolutional neural network (GCNN).

Alternatively or additionally, the target miRNA is miR-21.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram illustrating training of a deep learning model configured for virtual screening of molecules for micro ribonucleic acid (miRNA) drug discovery according to implementations described herein.

FIG. 2 is Table 1, which is a summary of the datasets used for bioactivity classification, side-effect prediction, and molecule selection according to an example described herein.

FIG. 3 is Table 2, which contains optimum hyper-parameters for each training scenario of the virtual screening models according to an example described herein.

FIG. 4 is a graph illustrating the AP scores of different sub-models for the task recommender algorithm according to an example described herein. There are 7 tasks that score higher than the threshold and thus are selected for training a new model. These tasks are elaborated upon in Table 3.

FIG. 5 is Table 3, which contains the recommended and optimized tasks for multitask learning for miR-21 virtual screening according to an example described herein. Each assay in Table 3 is represented by its Assay Identifier (AID) in PubChem.

FIG. 6 is Table 4, which contains the performance of different models on the test set of the miR-21 dataset according to an example described herein.

FIGS. 7A-7C illustrate the confusion matrix for models trained for single task (FIG. 7A), multitask for all tasks (FIG. 7B), and multitask for recommended tasks (FIG. 7C) according to an example described herein.

FIG. 8 is Table 5, which contains performance of toxicity and dicer activity models on their corresponding test sets according to an example described herein.

FIGS. 9A-9C are UMAP of the inner features of the model for the inference and training data according to an example described herein. FIG. 9A is for ZINC data predicted to be active. FIG. 9B is the training data and the remainder of the inference data. FIG. 9C is the 10 clusters applied to this space.

FIG. 10 is an example computing device.

FIG. 11 is a block diagram illustrating a small molecule drug discovery platform (RiboStrike) according to an example described herein.

FIG. 12 is a block diagram illustrating the flow of data withing the GCNN model of the small molecule drug discovery platform of FIG. 11.

FIG. 13 is a gene set enrichment analysis to sort candidate molecules based on upregulating effect on miR-21-3p target genes.

FIG. 14 illustrates the results of a reporter assay designed to assess the activity of five selected candidate hits. Three of the candidates showed promising activity.

FIG. 15 illustrates the results of treating MDA-MB-231tr breast cancer cells for 48 hours with the most promising candidate molecule, MCULE-9082109585.

FIG. 16 is a table that illustrates the performance of RiboStrike's best model compared to ChemBERTa on the test set of the miR-21 dataset.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for deep learning-based methods for developing a model for micro-RNA 21, it will become evident to those skilled in the art that the implementations are not limited thereto, but are applicable for developing models for other miRNA relevant to oncology and/or models for discovering inhibitors or activators of micro-RNA important for multiple diseases.

As used herein, the terms “about” or “approximately” when referring to a measurable value such as an amount, a percentage, and the like, is meant to encompass variations of +20%, +10%, +5%, or +1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

Described herein are a deep learning training method and deep learning model that can predict the interaction between a given small molecule and a targeted miRNA. In the examples described herein, the targeted miRNA is miR-21 (a type of Oncomir), which is a very important player in oncology, and it has been shown that inhibiting it would fully regress some types of tumors [I. B. Weinstein, “Addiction to Oncogenes—the Achilles Heal of Cancer,” Science, vol. 297, no. 5578, pp. 63-64, 2002, doi: doi: 10.1126/science.1073096]. It should be understood that miR-21 is provided only as an example targeted miRNA. The trained model described herein is designed to identify any molecules capable of disrupting the targeted miRNA activities through inhibiting the targeted miRNA itself, or inhibiting any protein upstream important for the targeted miRNA. The deep learning training method and deep learning model described herein address existing challenges associated with discovering small molecules for targeting miRNAs. Such challenges include, but are not limited to, the non-rigid structure of miRNAs, the small dynamic structures of miRNAs, and/or the lack of pockets and/or pockets of smaller sizes in miRNAs as compared to proteins. Moreover, structural binding of a small molecule to an miRNA may not impair the miRNA's functionality, and miRNA upstream modulators are generally unknown, each of which makes small molecule drug discovery challenging. For at least the above challenges, conventional drug discovery techniques such as SBDD have not been found useful for predicting the interaction between a given small molecule and a targeted miRNA. In contrast, the deep learning training method and deep learning model described herein provide solutions to the above challenges. For example, the trained model described herein (i.e., a model trained according to the deep learning training method described herein) works 100% non-structurally. As described herein, this means that the model does not need RNA structures to target them. The trained model described herein can therefore discover molecules with the ability of targeting miRNAs (inhibiting or activating), or any important upstream player in miRNA development including unknown proteins. The deep learning training method described herein also includes a task recommender that improves performance of the modelling, for example by addressing challenges associated with negative transfer learning. Additionally, the small molecule discovery platform described herein is completely automated and end to end, meaning that it just requires high-throughput screening (HTS) molecular data for training. Afterward, it can screen any number of molecules to discover their bioactivity against miRNA.

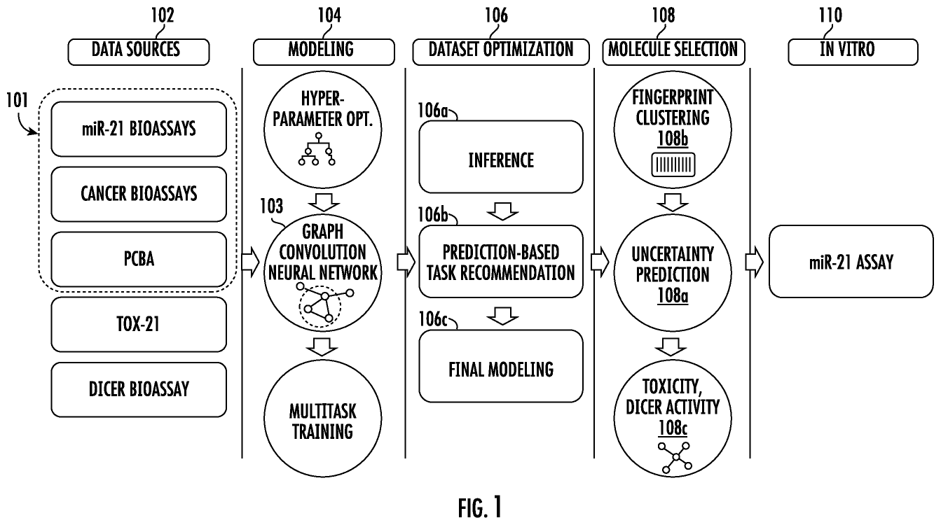

Referring now to FIG. 1, a method for training a deep learning model configured for virtual screening of molecules for miRNA drug discovery is shown. As described herein, the deep learning model training method uses multitask learning. Additionally, the deep learning model training method uses a task based recommender, which addresses issues of negative transfer for multiclass learning. In the examples described herein, the target miRNA is miR-21 (sometimes referred to as “miR21” or “miRNA21” or “microRNA 21” or “micro-RNA 21”), which is a mammalian microRNA encoded by the MIR21 gene. As noted above, miR-21 is an important player in oncology. miR-21 is frequently upregulated in solid tumors, and inhibition of miR-21 has been shown to fully regress some types of tumors. It should be understood that miR-21 is provided only as an example target miRNA. This disclosure contemplates that the target miRNA may be miRNA other than miR-21, which may include, but are not limited to, miR-22, which is relevant to liver disease and oncology.

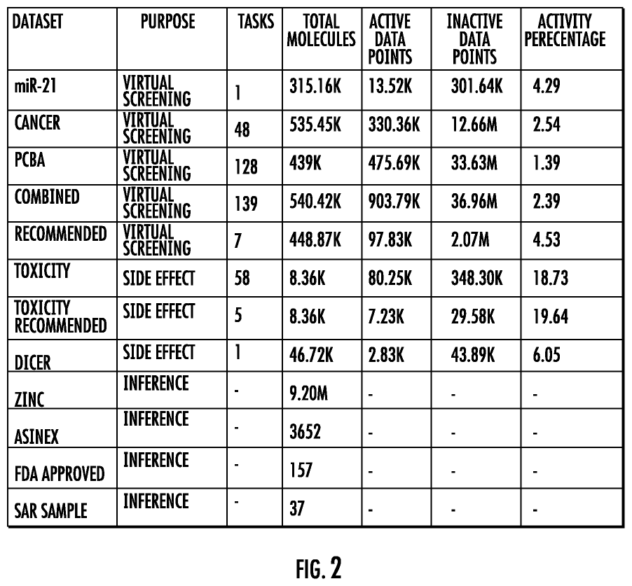

At step 102, the method includes creating a training dataset 101 including a plurality of assay datasets. The plurality of assay datasets include at least one miRNA assay dataset such as a miR-21 dataset. As described in the Example below (see e.g., FIG. 2), the plurality of assay datasets also optionally include cancer-related datasets (e.g., from PubChem or PubChem BioAssay (PCBA)) and/or PCBA datasets. It should be understood that miR-21 datasets, cancer-related datasets, and PCBA datasets are provided only as examples. This disclosure contemplates that the training dataset can include other assay datasets. Additionally, each of the assay datasets includes a plurality of molecules expressed in a computer-readable format. Optionally, the computer-readable format can be Simplified Molecular Input Line Entry System (SMILES) notation. SMILES notation is a line notation known in the art that uses ASCII strings. It should be understood that SMILES notation is provided only as an example and that other line notations can be used.

At step 104, the method includes training the deep learning model to learn a plurality of tasks using the training dataset. As described in the Example below, each of the plurality of assay datasets is associated with a respective task. Thus, the deep learning model is trained for a respective task using each of the datasets. This is known as multitask learning, where multiple tasks are learned by a shared model (e.g., deep learning model 103). As described in the Example below, multitask learning results in learned representations for one or more of the tasks benefiting the shared model. However, training using one or more of the datasets may have a negative impact (e.g., negative transfer) on the shared model. Accordingly, the method described herein includes a task recommender that is designed to address the problem of negative transfer (i.e., where multitask learning for a given task has a negative impact on the shared model).

In some implementations, the deep learning model 103 is an artificial neural network (ANN), which is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanH, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation.

Optionally, the deep learning model 103 is a graph convolutional neural network (GCNN). GCNNs are convolutional neural networks (CNNs) that have been adapted to work on structured datasets such as graphs. A CNN is a type of deep neural network that has been applied, for example, to image analysis applications. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks.

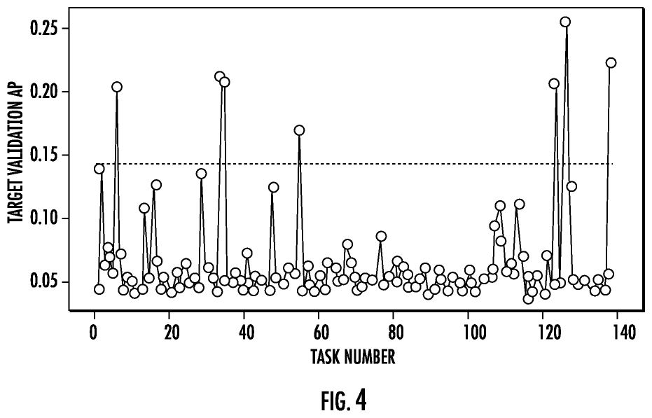

At step 106, the method includes performing, using the deep learning model, inference for the respective task associated with the at least one miRNA assay dataset, where the deep learning model outputs a respective prediction for each of the plurality of tasks (see step 106a); evaluating respective performance metrics associated with the respective predictions for each of the plurality of tasks; selecting a set of the plurality of assay datasets for training based on the respective performance metrics (see step 106b); and training the deep learning model to learn a set of the plurality of tasks using the set of the plurality of assay datasets (see step 106c). Additionally, the step of evaluating respective performance metrics associated with the respective predictions for each of the plurality of tasks includes calculating respective scores based on a comparison of the respective predictions for each of the plurality of tasks to ground truth labels for the respective task associated with the at least one miRNA assay dataset (see e.g., Equation 1 in the Example below). Optionally, the set of the plurality of assay datasets are associated with respective scores greater than a threshold (see e.g., FIG. 4). Optionally, the threshold is the mean score plus two standard deviations. It should be understood that mean score plus two standard deviations is provided only as an example. This disclosure contemplates selecting a different threshold.

As described in the Example below, step 106 results in a prediction-based task recommendation, which is used for training the deep learning model. Such prediction-based task recommendation results in the deep learning model being trained for the subset of tasks (e.g., the 7 tasks scoring higher than the threshold shown in FIG. 4), which minimizes the impact of negative transfer. For example, the deep learning model is trained in a multitask manner (see step 104) using the training dataset, which includes a plurality of assay datasets (see step 102), each of which is associated with a respective task. Thereafter, at step 106a, a target task is selected (e.g., the miR-21 dataset) and inference is performed on a validation dataset for the target task. Since the deep learning model was trained on multiple tasks, the deep learning model outputs a plurality of predictions for each input molecule, each assigned to one input task. This results in an M×N matrix of predictions, with each row representing the output of the sub-model assigned to a respective task. At step 106b, the deep learning model's performance is evaluated to identify a set of the plurality of assay datasets for final model training. For example, each task can be scored using Equation 1 in the Example below. It is important to note that the ground truth label (Label_{target}) is kept constant on the target task (e.g., the miR-21 dataset) while the sub-models change such that the predictions of the respective sub-models are compared to ground truth for the target task (e.g., the miR-21 dataset). The sub-models that have more similar predictions to the labels for the target task have higher scores, which facilitates identification and selection of sub-models (i.e., the recommended tasks) with similar predictions for the target task. At step 106c, the deep learning model is trained to learn a set of the plurality of tasks using the set of the plurality of assay datasets. In other words, at step 106c, the deep learning model is trained to learn a set of tasks that have been found to score higher than a threshold level of performance. This ensures that multitask learning results in learned representations that benefit the deep learning model.

As a result, the trained deep learning model is configured to predict molecules capable of affecting the target miRNA. In some implementations, a molecule predicted to affect the target miRNA directly inhibits the miRNA target. For example, the predicted molecule may interact with and/or disrupt activities of the target miRNA. In other implementations, a molecule predicted to affect the target miRNA has an indirect effect on the target miRNA. For example, the predicted molecule may influence a pathway related to the target miRNA such as a protein upstream of the target miRNA. The trained deep learning model can be used for screening of molecules for miRNA drug discovery. For example, at step 108, the method optionally includes inputting an inference dataset into the trained deep learning model; and predicting, using the trained deep learning model, a plurality of molecules capable of interacting with the target miRNA. Additionally, at step 110, the method optionally includes performing experimental validation such as in vitro testing on at least one of the plurality of molecules predicted as capable of affecting the target miRNA.

In some implementations, the method optionally further includes providing respective uncertainty scores for each of the plurality of molecules predicted as capable of interacting with the target miRNA; and selecting the at least one of the plurality of molecules on which experimental validation such as in vitro testing is performed based, at least in part, on the respective uncertainty scores. This is illustrated by reference number 108a in FIG. 1. It should be understood that step 108a can be performed before experimental validation at step 110. As described in the Example below, uncertainty scores can be calculated using the output of the last layer of the deep learning model (logits), where Dirichlet distribution is assumed for this output and uncertainty in regard to this distribution is calculated.

Alternatively or additionally, in some implementations, the method optionally further includes clustering the plurality of molecules predicted as capable of interacting with the target miRNA into a plurality of clusters; and selecting the at least one of the plurality of molecules on which experimental validation such as in vitro testing is performed based, at least in part, on at least two of the plurality of clusters. This is illustrated by reference number 108b in FIG. 1. This disclosure contemplates that step 108b can be performed before experimental validation at step 110.

Alternatively or additionally, in some implementations, the method optionally further includes inputting the plurality of molecules predicted as capable of interacting with the target miRNA into a first machine learning model; predicting, using the first machine learning model, respective toxicity metrics for the plurality of molecules predicted as capable of interacting with the target miRNA; and selecting the at least one of the plurality of molecules on which experimental validation such as in vitro testing is performed based, at least in part, on the respective toxicity metrics. This is illustrated by reference number 108c in FIG. 1. In other words, in these implementations, toxicity is used as a metric for molecule selection.

Alternatively or additionally, in some implementations, the method optionally further includes inputting the plurality of molecules predicted as capable of interacting with the target miRNA into a second machine learning model; predicting, using the second machine learning model, respective dicer activity metrics for the plurality of molecules predicted as capable of interacting with the target miRNA; and selecting the at least one of the plurality of molecules on which experimental validation such as in vitro testing is performed based, at least in part, on the respective dicer activity metrics. This is illustrated by reference number 108c in FIG. 1. In other words, in these implementations, dicer activity is used as a metric for molecule selection.

A method for virtual screening of molecules for micro ribonucleic acid (miRNA) drug discovery is also described herein. The method includes providing a trained deep learning model. This disclosure contemplates that the deep learning model can be trained as described herein, for example according to the method of FIG. 1. Such a trained deep learning model is then deployed in inference model to predict a molecule capable of interacting with a target miRNA. The method further includes inputting a molecule into the trained deep learning model, wherein the molecule is expressed in a computer-readable format; and predicting, using the trained deep learning model, that the molecule is capable of interacting with a target miRNA. Additionally, structural information about the target miRNA is not a feature input into the trained deep learning model. Alternatively or additionally, the trained deep learning model is a graph convolutional neural network (GCNN).

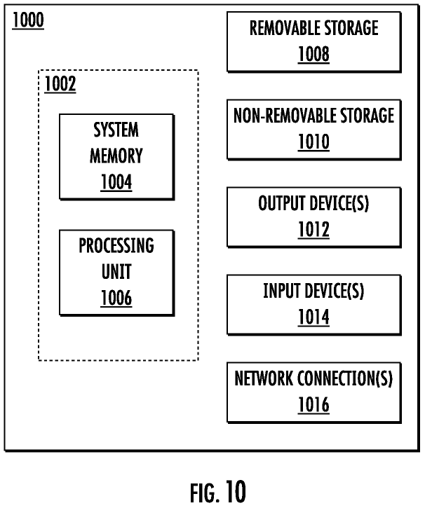

It should be appreciated that the logical operations described herein with respect to the various figures may be implemented (1) as a sequence of computer implemented acts or program modules (i.e., software) running on a computing device (e.g., the computing device described in FIG. 10), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 10, an example computing device 1000 upon which the methods described herein may be implemented is illustrated. It should be understood that the example computing device 1000 is only one example of a suitable computing environment upon which the methods described herein may be implemented. Optionally, the computing device 1000 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 1000 typically includes at least one processing unit 1006 and system memory 1004. Depending on the exact configuration and type of computing device, system memory 1004 may be volatile (such as random access memory (RAM)), non-volatile (such as read-only memory (ROM), flash memory, etc.), or some combination of the two. This most basic configuration is illustrated in FIG. 10 by dashed line 1002. The processing unit 1006 may be a standard programmable processor that performs arithmetic and logic operations necessary for operation of the computing device 1000. The computing device 1000 may also include a bus or other communication mechanism for communicating information among various components of the computing device 1000.

Computing device 1000 may have additional features/functionality. For example, computing device 1000 may include additional storage such as removable storage 1008 and non-removable storage 1010 including, but not limited to, magnetic or optical disks or tapes. Computing device 1000 may also contain network connection(s) 1016 that allow the device to communicate with other devices. Computing device 1000 may also have input device(s) 1014 such as a keyboard, mouse, touch screen, etc. Output device(s) 1012 such as a display, speakers, printer, etc. may also be included. The additional devices may be connected to the bus in order to facilitate communication of data among the components of the computing device 1000. All these devices are well known in the art and need not be discussed at length here.

The processing unit 1006 may be configured to execute program code encoded in tangible, computer-readable media. Tangible, computer-readable media refers to any media that is capable of providing data that causes the computing device 1000 (i.e., a machine) to operate in a particular fashion. Various computer-readable media may be utilized to provide instructions to the processing unit 1006 for execution. Example tangible, computer-readable media may include, but is not limited to, volatile media, non-volatile media, removable media and non-removable media implemented in any method or technology for storage of information such as computer readable instructions, data structures, program modules or other data. System memory 1004, removable storage 1008, and non-removable storage 1010 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1006 may execute program code stored in the system memory 1004. For example, the bus may carry data to the system memory 1004, from which the processing unit 1006 receives and executes instructions. The data received by the system memory 1004 may optionally be stored on the removable storage 1008 or the non-removable storage 1010 before or after execution by the processing unit 1006.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Methodology

### Overview

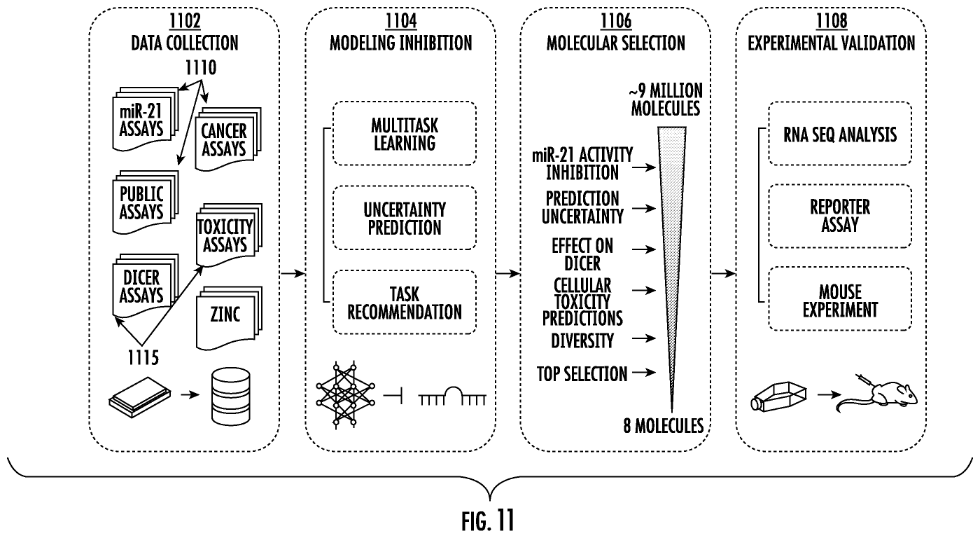

A small molecule drug discovery platform (referred to as “RiboStrike”), which focuses not on the structural inhibition of microRNAs, but rather on inhibiting their activity is shown in FIG. 11. The RiboStrike pipeline shown in FIG. 11 includes data aggregation and preprocessing 1102, modeling 1104, molecular selection 1106, and experimental validation 1108. Deep learning was chosen as the primary modelling tool because of the capabilities of deep learning to learn representations from molecular data and discover hidden patterns in an abstract and non-linear manner. For example, RiboStrike employs Graph Convolutional Neural Networks (GCNNs) (e.g., a deep learning model) to aid the virtual screening of small molecules against microRNA-21 solely relying on the graph-based input. In the modelling 1104 phase of the pipeline, multitask learning is used to learn patterns from multiple data sources including a reporter assay to discover candidate molecules with inhibitory effects on microRNA-21, assays for inhibiting selected cancer targets activity, and other large publicly available assays from PubChem. Various data sources are illustrated in the data collection 1102 phase of the pipeline. By using multiple predicted metrics such as uncertainty of activity, general toxicity, specific toxicity by an effect on dicer, and molecular diversity, molecules are cherry-picked in the molecular selection 1106 phase of the pipeline for wet lab screening in the experimental validation 1108 phase of the pipeline. For example, during inference, RiboStrike relies on its trained models to select 8 candidates from a pool of more than 9 million molecules of the ZINC database. RiboStrike discovers three candidate drugs validated experimentally with two in-vitro assays and a mice study. By using deep learning, RiboStrike proves that the discovery of candidate inhibitory molecules may be achieved even without structural information. Additionally, the RiboStrike pipeline includes a prediction-based algorithm for recommending input datasets for multitask learning in order to improve the performance of the modeling for the discovery of molecules that inhibit microRNA-21 activity.

The pipeline described herein can include, but is not limited to, data aggregation and preprocessing, modeling, training task optimization, molecular selection, and finally experimental validation such as in vitro screening. Example pipelines are illustrated by FIGS. 1 and 11. The data is aggregated for multiple assays including miR-21, cancer related assays, as well as a plethora of unrelated assays. Moreover, data is gathered for side-effect minimization, including toxicity and dicer activity. After preprocessing, the training set is used to train a Graph Convolutional Neural Network (GCNN) in a multitask manner, with validation set used for hyper-parameter optimization and best epoch determination. After the multitask model is trained on all the data, training tasks are limited and chosen using the prediction-based inference algorithm. The final model is trained on the recommended dataset, and inference is performed on multiple datasets including ZINC and LINCS datasets. The final molecules are selected based on bioactivity prediction towards miR-21, uncertainty of this predictions, toxicity predictions, dicer activity prediction, and diversity. The top “n” selected molecule are then experimentally validated (e.g., tested in vitro). In this Example, eight molecules from a pool of more than 9 million molecules of the ZINC database are selected for experimental validation.

### Data Sources

### Virtual Screening Training Datasets

There are three sets of data used in this Example to train and validate virtual screening models: virtual screening datasets, off-target interaction datasets, and inference datasets. Each dataset is preprocessed to contain canonical SMILES as well as appropriate binary labels for the activity of molecules. These datasets are as follows:

miR-21 Data: The main data used for this Example is a HTS assay dataset on miR21 as a target. In this assay from PubChem, a data repository from the National Institutes of Health (NIH) and national laboratory of medicine. The ID of this data in PubChem is AID 2289. The aim of this assay had been inhibitory of the miR21 so induction of cell apoptosis and tumor suppression. They used a cell-based firefly luciferase reporter gene assay optimized for qHTS.

Cancer-Related Data: To assist the multitask training process, different cancer-related assays are collected from PubChem and the PubChem BioAssay (PCBA) dataset. These assays include 20 tasks directly from PubChem dataset, and 38 cancer related tasks from PCBA dataset. The 20 tasks from PubChem were selected if they had more than 300,000 tested molecules with a scope for cancer drug discovery.

PCBA dataset: PCBA is a collection of datasets aggregated from PubChem consisting of the biological activities of small molecules generated by high-throughput screening. In this Example, a subsection of PCA with 128 bioassays is used with over 400,000 molecules, similar to the previous benchmarking methods [B. Ramsundar, S. Kearnes, P. Riley, D. Webster, D. Konerding, and V. Pande. “Massively multitask networks for drug discovery.” arXiv preprint. arXiv: 1502.02072 (accessed May 18 2020)]. This dataset was selected due to its size, high number of tasks, and high molecular overlap with the miR-21 dataset. Due to these features, this dataset can be combined with the miR-21 dataset to create a large training set for multitask learning.

Overall, the three sets of data (miR21, Cancer, and PCBA) were collected, preprocessed and merged to create the “combined” dataset. Later in the process, through the task recommendation system (Optimizing the Tasks for Multitask Learning Section below), a subset of the tasks (i.e., less than all of the tasks) were selected from the combined dataset to assist the multitask training network. This dataset is referred to as the “recommended” dataset, and possesses fewer tasks than the combined dataset, which were selected with an algorithmic approach.

### Side Effect Datasets: Toxicity and Dicer Activity

To increase the probability of any predicted bioactive molecule to be a drug candidate, toxicity and Dicer activity of these molecules are predicted. Dicer is a very important protein for maturation of many vital RNAs, not just miR21. Therefore, if the candidate drug inhibits the miR21 by actually inhibiting the Dicer protein, it will show significant side effects since it is inhibiting basically every RNA. On the other hand, there are a lot of public datasets for showing the toxicity of molecules like Tox21. It consists of at least 50 distinct tasks each tested on an important protein target of humans. Any candidate drug showing interaction with those targets would show probable side effects and toxicity in the body. Therefore, 58 tasks from Tox21 were used to filter out molecules that would show side effects in future steps. The two datasets used in this Example for this aim are as follows:

Tox-21 (Toxicity in the 21st century) is from PubChem data source (https://pubchem.ncbi.nlm.nih.gov/source/824). Tox21 has been an effort from NIH to test roughly 10,000 molecules on important protein targets in the body. This dataset consists of 58 distinct tasks each tested on an important protein target of humans. Candidate drugs that interact with those targets are likely to cause side effects and cellular toxicity. Therefore, 58 tasks from Tox21 are used to filter out molecules that would show side effects in future steps.

Dicer dataset: The Dicer protein plays an important role in the maturation of several RNAs, not just miR21. Therefore, if a candidate drug decreases the activity of the miR21 by inhibiting the Dicer protein, it would show significant side effects downstream. Dicer data base is from PubChem data source of AID 1347074 (https://pubchem.ncbi.nlm.nih.gov/bioassay/1347074). This dataset was performed by a technic called click chemistry. This dataset was chosen since they used pre-miR21 to run this assay and identify Dicer inhibitors.

### Inference Data

There are multiple datasets used in this Example for selection of molecules:

ZINC: ZINC database is a useful molecular library for virtual screening. It contains millions of molecules which are mostly purchasable. A set of 9 million molecules from ZINC database were used for screening. For example, the drug-like subset (MW: 250˜500, LogP: −1˜5) of the ZINC15 database, consisting of 9 million molecules that had 3D representations, standard reactivity, reference pH, a charge of −2 to +2, were used. These 9 million molecules were chosen due to their draggabilities.

LINCS: The library of integrated network-based cellular signatures (LINCS) is a library from NIH that catalogs the changes that agents do to disrupt normal cellular functions. Most of LINCS molecules are available to buy.

Asinex: Asinex is a molecular library vendor that cells varieties of different classes of molecules including macrocycles, alpha helix mimetics and peptidomimetics. It has a library called RNA-targeting small molecules which was suitable for the purposes of this Example.

FDA Approved: This dataset consists of FDA approved molecules screened on miR-21 [S.-R. Ryoo et al., “High-throughput chemical screening to discover new modulators of microRNA expression in living cells by using graphene-based biosensor,” Scientific Reports, vol. 8, no. 1, p. 11413, 2018 Jul. 30, 2018, doi: 10.1038/s41598-018-29633-x]. This resulted in 86 molecules that were bioactive against miR-21. In this Example, these molecules are deleted from the mentioned inference datasets, to avoid selection of previously discovered molecules. Moreover, these molecules were used to assist the selection of the final molecules as described in the Molecule Selection section below.

SAR Sample: This dataset originates from a study which takes two molecules that are known inhibitors of miR21, and uses Structural Activity Relationship (SAR) to optimize these molecules and assist in overcoming chemoresistance in Renal Cell Carcinoma. Overall, 37 molecules are tested and shown to be active, which was used to create a small validation set for this Example.

### Molecular Data Preprocessing

After gathering multiple datasets for bioactivity classification, side effect prediction, and inference, the molecules are represented in simplified molecular input line entry system (SMILES) format. Each entry is the transformed to be canon with isomeric information included, using rdkit library in Python (v3.6). Afterwards, the data is cleaned and missing, or duplicate, entries are removed. The molecules from the toxicity dataset undergo one more preprocessing step, during which the molecules were desalted, and inorganic molecules were removed. This is since many molecules exist in the toxicity dataset that salted, redundant or contains inorganic atoms. Since these molecules does not apply to this project, these molecules are removed.

### Label Assignment

In order to assign bioactivity labels to each molecule from the PubChem datasets, the two columns of “outcome activity” and “phenotype activity” were examined. In most assays, the datapoints with “Activator” phenotype were removed, since just the inhibitors were wanted but not the activators which confuse the modeling. In two cases (AIDs 504466 and 624202) the inhibitors were removed instead, since inhibitor and activators had the opposite meaning. For the remaining molecules, the outcome activity column is used to assign bioactivity labels, with ‘Active’ and ‘Inconclusive’ outcomes being labeled as 1, and ‘Inactive’ and ‘Unspecified’ outcomes being labeled as 0.

### Data Splitting

The combined dataset, toxicity dataset, and dicer dataset are all split into training (80%), validation (10%), and test sets (10%) using DeepChem [B. Ramsundar, P. Eastman, P. Walters, V. Pande, K. Leswing, and Z. Wu, Deep Learning for the Life Sciences. O'Reilly Media, 2019], based on the molecular scaffolds [G. W. Bemis and M. A. Murcko, “The Properties of Known Drugs. 1. Molecular Frameworks,” Journal of Medicinal Chemistry, vol. 39, no. 15, pp. 2887-2893, 1996 Jan. 1, 1996, doi: 10.1021/jm9602928]. Splitting based on the scaffolds allows a larger distinction to exist between the molecules of each split, resulting in a more practical model for real-world applications where inference data is often from a different distribution than the training data. Moreover, splitting the combined dataset instead of each of the virtual screening datasets allows for the test dataset to remain the same for each model trained on each of the main datasets, resulting in a fair comparison between the models.

### Graph Convolutional Neural Network Training

In the recent years GCNNs have proven to be helpful in learning representations from small molecules and modeling tasks such as virtual screening [M. Sun, S. Zhao, C. Gilvary, O. Elemento, J. Zhou, and F. Wang, “Graph convolutional networks for computational drug development and discovery,” Briefings in bioinformatics, vol. 21, no. 3, pp. 919-935, 2020], molecular property prediction [M. Sakai et al., “Prediction of pharmacological activities from chemical structures with graph convolutional neural networks,” Scientific Reports, vol. 11, no. 1, p. 525, 2021 Jan. 12, 2021, doi: 10.1038/s41598-020-80113-7], and drug-target interaction [W. Torng and R. B. Altman, “Graph Convolutional Neural Networks for Predicting Drug-Target Interactions,” Journal of Chemical Information and Modeling, vol. 59, no. 10, pp. 4131-4149, 2019 Oct. 28, 2019, doi: 10.1021/acs.jcim.9b00628; T. Zhao, Y. Hu, L. R. Valsdottir, T. Zang, and J. Peng, “Identifying drug-target interactions based on graph convolutional network and deep neural network,” Briefings in bioinformatics, vol. 22, no. 2, pp. 2141-2150, 2021]. This success is owed to two facts, firstly small molecules are inherently similar to graphs, with atoms represented as nodes and bonds represented as edges, therefore, GCNNs are suitable tools for application on this data type. Secondly, the feature extraction in the GCNN model, which is inspired by traditional circular fingerprint extraction from molecules, results in useful and often superior inner features due to the automatic representation learning aspect of deep learning [D. K. Duvenaud et al., “Convolutional networks on graphs for learning molecular fingerprints,” in Advances in neural information processing systems, 2015, pp. 2224-2232; S. Kearnes, K. McCloskey, M. Berndl, V. Pande, and P. Riley, “Molecular graph convolutions: moving beyond fingerprints,” Journal of Computer-Aided Molecular Design, vol. 30, no. 8, pp. 595-608, 2016 Aug. 1, 2016, doi: 10.1007/s10822-016-9938-8].

In this Example the GCNN implementation from the DeepChem library is used. In this model the molecules are converted to graphs and atoms are featurized to include features such as atom type, number of directly bonded neighbors, implicit valence, formal charge, and hybridization type. Isomeric information is also added to the features in the form of a vector with length of 3 (whether chirality is possible, right-hand, and left-hand). The hyper-parameters of this model as well as the length of the training are found through hyper-parameter optimization in a grid search manner.

### Hyper-Parameter Optimization and Training

After feature extraction an optimum model architecture needs to be identified which can deliver optimum performance given the training dataset. Using the validation set, different model architectures are evaluated for their performance. In a grid search manner, the number of graph convolutional layers, the size of each layer, the size of the feed forward layer, the batch size, and the learning rate are altered and the best performing set of parameters on the validation set is taken to initiate the training. This hyper-parameter optimization is extended to the training epoch selection, where the epoch with the best performance on the validation set is chosen as the final model.

### Evaluation Metric

The metric used for evaluation in this Example is the average precision (AP) score. This metric computes the area under the precision-recall curve and was chosen due its fairness towards imbalanced datasets, where the positive label discovery is of importance. This is the case with most virtual screening tasks, where the number of active molecules is often much lower than the number of inactive molecules, resulting in highly imbalanced datasets. Moreover, discovery of these active candidates is of utmost importance in an early drug discovery pipeline since these candidates will be passed on to the next steps of the drug discovery process (e.g., experimental validation). Therefore, average precision score is favored in this Example for comparison of models, different architectures, or different epochs during training. The results are also reported for accuracy, recall, precision, and the area under Receiver Operator Curve (ROC-AUC).

### Optimizing the Tasks for Multitask Learning

Multitask learning has proven to be beneficial in many instances via providing multiple tasks for the model to simultaneously learn from, with the hope that the learned representations for these tasks benefit from being shared within the same model. However, this is not the case in all scenarios and in some cases negative transfer occurs, where multitask learning hurts the performance of a given task when compared to single task learning. In this Example, to address the problem of negative transfer, the method of “prediction-based task recommendation” is used, which narrows the number of tasks selected for multitask learning via recommending a few tasks in an algorithmic manner.

### Prediction Based Task Recommendation

To begin the process of task recommendation and selection of fewer training tasks, a multitask learning model is trained on all available tasks. After training, one target task is selected (i.e., miR-21 dataset) and inference is performed on the validation set of this target task. Since the model is trained on multiple tasks, it will have multiple predictions for each input molecule, each assigned to one input task. Given N total training tasks and M molecules in the target task's validation set, the predictions of the model will then have a shape of M×N, with each row representing the output of the sub-model assigned to one input task, denoted by Outputᵢ. After this output is calculated, each task i is scored using the scoring metric in Equation 1.

Scoreᵢ=Average_Precision_Score(Label_{target},Outputᵢ) Equation 1

In which Label_{target} denotes the ground truth labels for the target task. As it can be seen from this equation, the labels are kept constant on the target set while the sub-model changes, which is the main difference between this method and simple inference. Using this scoring mechanism, the predictions of different sub-models are compared to the ground truth labels of the target task, with the sub-models that have more similar predictions to the target labels having a higher score. Through identification of sub-models with similar predictions, this approach identifies their corresponding training tasks and selects the highest scoring tasks for training. The recommended tasks are selected via applying a threshold of mean plus two standard deviations to the scores. This threshold is arbitrary and can be replaced with a simple selection of top K scores. The recommended tasks are then passed on as training data to the hyper-parameter optimization and training step for the final model. This recommendation process is repeated for the toxicity model as well, with the target task of HepG2.

### Molecule Selection

### Inference and Uncertainty Principals

After training is finished, the final model is used to predict the bioactivity of molecules from the inference datasets. All training data, as well as the FDA approved data, is removed from the inference datasets to avoid selection of redundant molecules. During the inference process, each molecule passes through the preprocessing step and the trained model and in the end a binary bioactivity prediction is achieved. Due to the large size of the inference data (approximately 9 million compounds) many molecules are predicted to be active, which is challenging due to multiple reasons. Firstly, the number of molecules that can be screened in vitro are orders of magnitude smaller than the number of molecules within the inference set. Secondly, the predictions are binary in which case two molecules that are predicted to be active are indistinguishable. Lastly, similar molecules have similar prediction, which in the case of ZINC dataset becomes a problem due to the existence of many similar molecules.

In order to overcome the selection challenges, firstly an uncertainty prediction method is applied on the last layer of the model. To do so, evidential deep learning is used [M. Sensoy, L. Kaplan, and M. Kandemir, “Evidential deep learning to quantify classification uncertainty,” arXiv preprint arXiv: 1806.01768, 2018] which applies a Dirichlet distribution on the class probabilities and computes uncertainty for each prediction. This uncertainty scores ranges from zero to one, with lower scores demonstrating more certain predictions. With this uncertainty score, the predictions become distinguishable and the molecules that are predicted to be active with low uncertainty become desirable.

### Neural Fingerprints Clustering

After the uncertainty is predicted, one problem remains which is the lack of diversity in the most certain molecules that are predicted to be active. This is since similar molecules result in similar predictions, and the most certain predicted molecule and similar molecules to it populate the top of the certainty ranking. This creates a problem in the further stages and specifically in vitro screening, where diversity within the candidates is needed to increase the chance of bioactivity or activity against different targets.

To enforce diversity within the selected molecules and create variety within the final selection, the molecules are clustered, and a few molecules are selected from each cluster. To do so, the neural fingerprints of the molecules are extracted from the Graph Gather layer of the trained model. This fingerprint is the inner features of the model for the input molecule and is a vector which can meaningfully represent it. Neural fingerprint clustering allows the molecules belonging to different clusters to be both structurally different and exist in different locations within the feature space of the trained network. After the features are extracted, KMeans (K=10) clustering is applied to the 2D UMAP projection of the features, and 10 clusters are formed from the inference molecules.

### Criteria for Molecule Selection

After the molecules are clustered and uncertainty and bioactivity is predicted for all of them, five different criteria were met for the final molecules to be selected:

Potency as miR21 activity inhibitor: The selected molecules should be predicted to be inhibitors of miR-21.

Certainty: The molecules that had the least uncertainty in each cluster were considered.

Diversity: Each molecule should belong to a different cluster in regard to the clustering of the neural fingerprint. Clusters that include more of the FDA approved molecules are more likely to be selected from.

Pass majority of toxicity tests: The selected molecules should be predicted to be non-toxic in most of the toxicity tests with low uncertainty for specifically the HepG2 test.

Low chance of inhibiting the Dicer: The selected molecules should be predicted to not inhibit the dicer with low uncertainty.

Following these criteria, the inference molecules are first narrowed down to those predicted to be active with high certainty, then filtered via selecting top molecules from each 10 clusters. Afterwards, the final molecules are selected from this list with consideration of toxicity and dicer activity and their uncertainties. In the end, 8 molecules were selected from each of the inference datasets (ZINC and Asinex), and progress to the experimental validation stage. The same process is partially applied on the molecules that are predicted to have no bioactivity, and 5 molecules are selected for control which have no bioactivity with low uncertainty.

### Cell Culture

The MDA-MB-231 (MDA-parental, ATCC HTB-26) human breast cancer cell line; its highly metastatic derivative, MDA-LM2 [PMID 16049480]; its triple reporter version, MDA-MB-231tr; and HEK293T cells (ATCC CRL-3216) were cultured in Dulbecco's modified Eagle medium supplemented with 10% fetal bovine serum (FBS), penicillin, streptomycin, and amphotericin B. Cells were all incubated at 37° C. at 5% CO2 in a humidified incubator.

### Cell Titer-Glo Cell Viability Assay

MDA-MB-231 LM2 cells were seeded onto 96-well plates at 3×10\hat{ }3 cells per well. Cells were treated at a consistent range of concentrations from 3.2 pM to 1 mM over 3 days. Cell viability of samples with selected drug concentrations were assessed using the CellTiter-Glo cell viability assay (Promega) according to manufacturer's instructions. IC20 concentrations of these samples were determined using GraphPad (https://www.graphpad.com/quickcalcs/Ecanything1.cfm).

### High-Throughput Sequencing Data Generation

MDA-MB-231 LM2 cells were seeded at a density of 3×10³ cells per well of a 96-well plate, and treated at IC20 concentrations over 3 days. Additionally, anti-miR controls (anti-mi21 and non-targeting) were also transiently transfected into cells using Lipofectamine 2000 (ThermoFisher) according to the manufacturer's protocol. Cells were collected after the 3 day incubation period and RNA was extracted from the cell pellets with Zymo Quick-RNA 96 kit according to manufacturer's instructions. RNA-seq libraries were prepared using the QuantSeq Pool Sample-Barcoded 3′ mRNA-seq Library Prep Kit (Lexogen).

### RNA-Seq Data Analysis

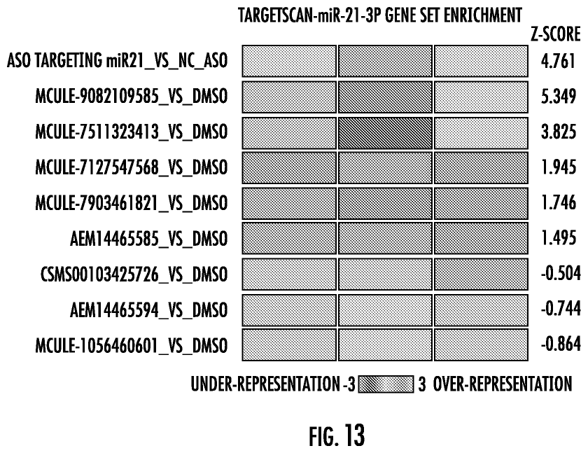

The QuantSeq Pool data demultiplexed and preprocessed using an implementation of the pipeline provided by Lexogen (https://github.com/Lexogen-Tools/quantseqpool_analysis). The outputs of this step are gene level counts for all samples. The raw counts matrix used for differential expression analysis using DESeq2 package [PMID 25516281]. The log fold changes from multiple differential expression comparisons used for making correlation matrix (see FIG. 13).

Finally, molecules were sorted based on their systematic effect on expression of microRNA-21 target mRNAs through gene set enrichment analysis. Thus, TargetScan prediction of hsa-mir-21 3p target genes were downloaded from miRBase dataset (Accession: MI0000077). Then, a modified version of iPAGE [PMID 20005852] (called onePAGE from here) was used to perform the gene set enrichment analysis powered by mutual information evaluation and statistical tests for a single gene set. The onePAGE analysis here reports enrichment of miR21 target gene list in 3 bins of log fold change values; from left to right 0) lowest bin of log 2FC, i.e., downregulated, 1) log 2FC around zero, i.e., no expression change. 2) highest bin of log 2FC, i.e., upregulated. After running this analysis for differential expression log 2FC of all drugs and control conditions, drugs were sorted based on resulted z-score with keeping miR21 versus negative control (nc) at the top (see FIG. 13). From this step, the top 6 drugs were selected for further evaluation.

Generation of MDA EGFP^{miR-21} reporter cell line

The vector backbone for the reporter plasmids was generated from a vector with a bi-directional CMV promoter driven lentiviral reporter, expressing eGFP and AlnGFR. This vector was a gift from David Erle [PMID: 15619618]. The ΔlnGFR ORF in the vector was replaced by a PuroR-T2A-mCherry fusion using Gibson assembly as previously described [PMID: 32513775]. In order to generate the eGFP^{miR-21} reporter plasmid two miR-21 binding sites were added to the end of eGFP using the NEBuilder HiFi DNA Assembly Cloning Kit. MDA-MD-231 cells were engineered to stably express the reporter plasmid using lentiviral delivery of the vector.

hsa-miR-21-5p
(SEQ ID NO: 1)
TCAACATCAGTCTGATAAGCTA

Sequences for Reporter Cell Line Generation were Obtained from miRBase
FLOW Cytometry Analysis of miR21 Reporter Cell Line

MDA EGFP^{miR-21} reporter cell lines were seeded at a density of 2×10\hat{ }4 cells per well of a 96-well plate. Cells were treated for 3 days with serial dilutions of the 6 most promising drugs (dilutions determined by IC20 curves). Additionally, anti-miR controls (anti-mi21 and non-targeting) were also transiently transfected into cells using Lipofectamine 2000 (ThermoFisher) according to the manufacturer's protocol. Cells were collected after 3 days and fluorescence output was measured on a BD FACSCelesta flow cytometer.

### Animal Studies

All animal studies were performed according to IACUC guidelines (IACUC approval number AN194337-01G). Age-matched female NOD scid gamma mice (Jackson Labs, 005557) were used for metastatic lung colonization assays. These assays were performed with MDA-MB-231tr cells which were seeded at 1.5×10⁵ cells in 2 wells of a 6-well plate on Day 1. After 24 hours 0.1 uM MCULE-9082109585 and 0.5% of DMSO were added dropwise to one well each. After 48 hours, MCULE-9082109585 and 0.5% of DMSO media was removed and the cells were prepped for tail vein injections. Cells were resuspended in 2 ml PBS and each mouse received 5×10⁴ cells/100 ul of PBS. Metastasis was measured by bioluminescent imaging (IVIS), and histology performed by hematoxylin and eosin (H&E) staining of lung tissue sections.

### Data Collection Summary

Three different categories of data are used to train RiboStrike for early hit detection as shown in FIG. 11. To begin with, virtual screening datasets are used to train models that predict the effect of a particular small molecule on the function of miR-21 or assist in boosting its performance. In this category, inhibitors of miR-21 activity (“miR-21 Data”), assays designed to identify cancer-fighting candidates (“Cancer-Related Data”), and the PCBA dataset are combined during data collection 1102 in FIG. 11. The “combined” datasets are labelled 1110. During modelling 1104 in FIG. 11, the combined dataset is narrowed down to 7 tasks using the prediction-based task recommender algorithm in order to create a focused training dataset that is beneficial for multitask learning. This is referred to as the “recommended” datasets. Additionally, the pipeline makes use of datasets that provide information on “side effects”, such as general cellular toxicity and dicer inhibition, to guide its prediction that it will not be toxic and should avoid interfering with dicer, one of the most common targets for off-target interactions. The side-effect datasets are labelled 1115. Finally, inference datasets (e.g., LINCS and Asinex) were used during molecular selection 1106 in FIG. 11 in order to find candidates for further screening during experimental validation 1108 in FIG. 11.

The summary of datasets is shown in Table 1 (FIG. 2).

The combined dataset includes the combination of miR-21 dataset, the cancer dataset, and the PCBA dataset. As shown in Table 1, the “combined” dataset includes 139 tasks. Using the prediction-based task recommender algorithm, this dataset is narrowed down to 7 tasks. This is referred to as the “recommended” dataset. The combined dataset, alongside the toxicity and dicer dataset created three different categories for training.

### Computational Pipeline and Training Results

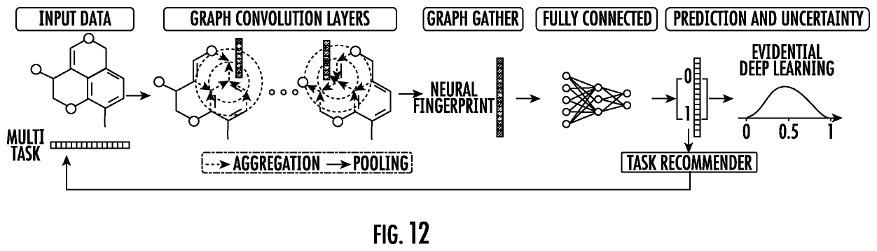

In learning from molecular data, RiboStrike utilizes GCNNs as its primary modeling method. Graph-based models are ideally suited for handling small molecules since nodes represent atoms within molecules and edges represent bonds between them. In this graph, each node contains features that describe the atom and its properties. Through the use of these features during training, an abstract representation of a molecule can be formed by passing messages between graph convolution layers and graph gathering layers. With the help of evidential deep learning, an estimate of the uncertainty of a prediction regarding the input molecule is calculated. To begin the RiboStrike process, models are trained to predict miR-21 activity suppression, dicer inhibition, and toxicity of given molecules. The performance of these models is further optimized by lowering the number of training tasks available via a task recommendation algorithm. FIG. 12 illustrates the pipeline for data flow in this example.

### Hyper-Parameter Optimization Results

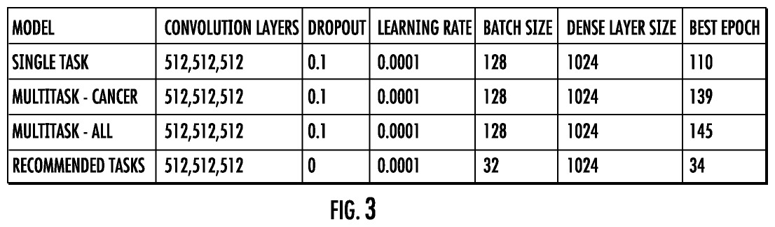

After the data is cleaned, hyper-parameter optimization is performed in a grid search manner and the results are shown in Table 2 (FIG. 3) for the virtual screening models. All models are trained for 150 epochs and the best epoch is identified using the AP score on the validation set.

Optimum hyper-parameter for each training scenario of the virtual screening models are shown in Table 2 (FIG. 3). As seen in Table 2 (FIG. 3), the hyper-parameter search arrives at similar optimum architectures for all datasets, with the model for the recommended task having a smaller batch size and training duration. This is intuitive since this training scenario has a lower number of tasks and a smaller training dataset.

### Task Recommendation Results

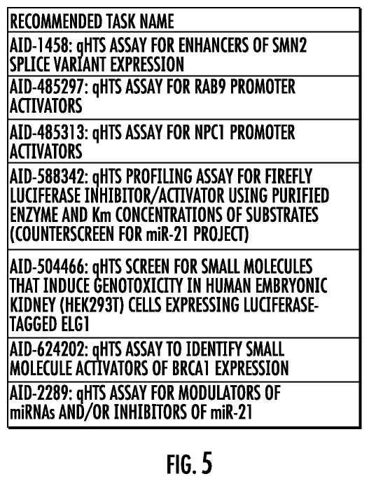

Despite the large dataset created by combining all assays, the number of tasks may adversely affect the model's performance. A particular problem may arise during multitask learning and Stochastic Gradient Descent (SGD) when some tasks differ from others regarding the gradient direction, which results in a less efficient training process. As a result, a prediction-based recommendation algorithm is used in this example to select a few tasks from the dataset and to create a smaller dataset after the multitask model has been trained on all tasks. Using this algorithm, sub-models with similar predictions to the target task (for example, miR-21) are identified, and tasks relating to these sub-models are identified and the top-ranking ones are selected as recommendations. FIG. 4 provides an overview of the AP scores for all tasks as well as the threshold line. As shown in FIG. 4, there are seven tasks that score higher than the threshold (of the mean plus two standard deviations) and are therefore recommended. Table 3 (FIG. 5) provides a detailed explanation of these tasks.

From Table 3 (FIG. 5), it can be seen that the counter screen for the miR-21 assay is positioned as one of the recommended tasks, which is intuitive due to its association with the main task and the importance of the information (molecular patterns and activity labels) contained in this dataset. However, even though the remaining tasks are not directly related to miR-21, the output of their trained sub-model is similar to that of the miR-21 sub-model. In other words, the models trained on these recommended tasks perform relatively better in predicting the effect on miR-21 activity than the rest of the sub-models, making their respective training data suitable for use in the recommended model.

### Virtual Screening Results

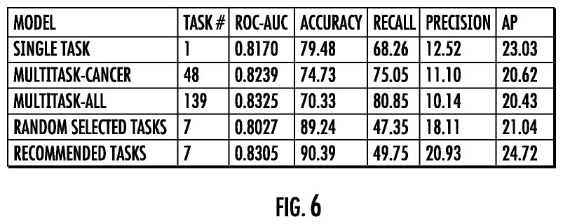

Following identification of the optimized tasks and implementation of all training scenarios, different modeling techniques can be compared to ascertain which dataset and training regimen resulted in the best-performing model. The results on the test set of the miR-21 dataset, which is kept constant and isolated throughout all models, are calculated and used to compare the performance of different training regiments. These results are shown in Table 4 (FIG. 6).

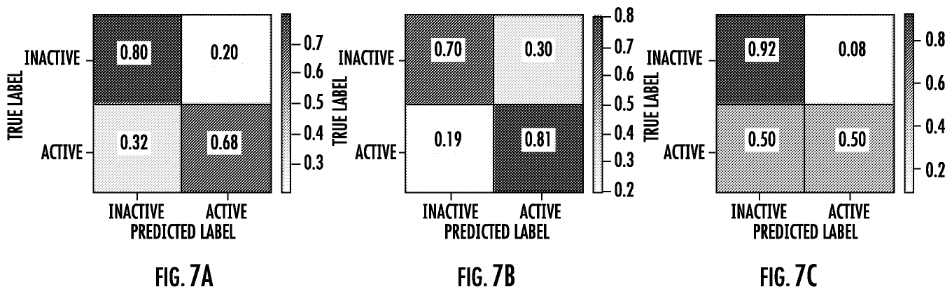

The prediction-based task recommender algorithm, as shown in Table 4 (FIG. 6), results in a better model for the recommended tasks than for random tasks, all tasks, or single tasks, and the model trained on the recommended tasks achieves the highest average precision score. Because this model has a lower tendency to predict molecules as active, it suffers from a loss in recall. It is evident, however, from the precision score in Table 4 (FIG. 6) and the confusion matrix in FIG. 7C, that molecules that are predicted to be active are more likely to actually be active.

A further investigation of the best-performing model is conducted using the Structural Activity Relationship sample dataset, which includes 37 molecules derived from two inhibitors of miR-21 that can overcome chemotherapy resistance in cancers such as renal cell carcinoma. It would be ideal if a virtual screening model could predict all 37 molecules as positive. As a result of the trained model, 33 out of 37 molecules were predicted as active, closely matching the results of the related study, demonstrating the potential for this model in the context of SAR scenarios. For the four misclassified molecules, the model outputs 100% uncertainty, the majority of which are in the last iteration of the SAR, where the original molecules have been significantly altered. Consequently, these molecules are outside the familiarity zone of the model's training set, resulting in an uncertain prediction for the model.

### Toxicity and Dicer Modeling Results

It is imperative that candidates for drug development are non-toxic and free from interactions with random targets on the cellular level. Among the main targets of miR-21, dicer is one whose inhibition can lower miR-21 activity, but also interfere with the activity of all micro RNAs. As a result, three models have been trained to predict the toxicity and inhibitory activities against the dicer as a way to identify any unwanted inhibitory effects on selected targets that could lead to cell toxicity.

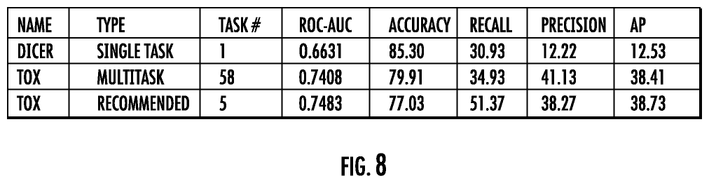

In order to determine molecular toxicity against cell viability, the HepG2 cell line was selected as a standard model. Moreover, one model was trained on the Tox21 dataset, which included 58 different toxicity tasks, with the purpose of predicting potential off-target interactions with important cellular components. Overall, the molecules that have fewer toxic predictions (out of 58) with lower uncertainty on HepG2 toxicity prediction are more desirable. The performance of these three models is depicted in Table 5 (FIG. 8).

For the case of dicer inhibition prediction, a single task model is trained on the available data. On the other hand, the toxicity dataset contains multiple assays, which requires the same training pipeline (multitask learning as well as task recommendation) as the miR-21 virtual screening model to be reimplemented. In this process after a model is trained on all of the toxicity tasks, the prediction-based recommendation algorithm returns 5 tasks that would assist the performance of the model on the HepG2 target task.

As it can be seen from Table 5 (FIG. 8), the model trained on the recommended tasks is able to predict HepG2 toxicity with a higher AP. Therefore, this model is used for predicting HepG2 toxicity and uncertainty, while the multitask model is used to predict the rest of the toxicity tasks. Molecules that have fewer toxic predictions (out of 58) with lower uncertainty on HepG2 toxicity prediction are more desirable.

### Inference and Clustering Results

Once the virtual screening model and side-effect model have been trained, they can be used to screen novel molecules for their potential as drug candidates. The choice of libraries for screening is ZINC, due to its large size and diversity, and Asinex, because of its libraries of specialized molecules. A trained model screens the datasets for suitable molecules to move on to the next step in drug discovery. As a first step, the molecules are fed into the multitask virtual screening model that is trained based on the recommended task to predict miR21 activity inhibition.

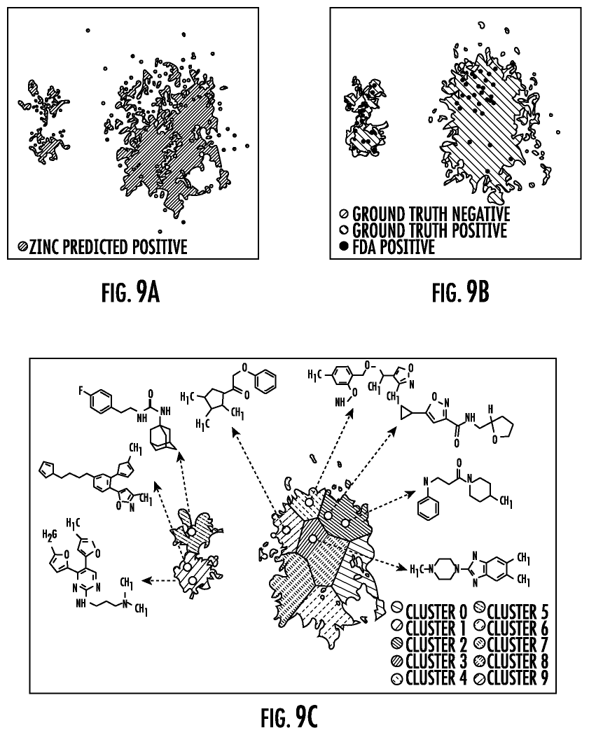

To obtain a more complete understanding of how diverse the inference molecules are, the molecular solace needs to be clustered. In addition to enhancing the diversity of selected molecules, this clustering can assist in selecting molecules from various regions of the molecular space. To accomplish this, the inner features of the trained model are extracted, projected into a 2D Uniform Manifold Approximation and Projection (UMAP) space, and clustered using the KMeans (also referred to as “k-means”) algorithm. k-means clustering is a clustering technique that is known in the art. By separating molecules into clusters, different regions of the molecular space can be accessed and diverse molecules can be selected for in vitro testing. FIGS. 9A-9C illustrate the result of clustering.

The positive and negative training data distributions shown in FIG. 9B have a substantial overlap, presenting a challenging scenario when screening for miR-21 using virtual screening. It is also evident from FIG. 9A that the molecules from the ZINC database that have been predicted to be positive cover most of the projected space, demonstrating the diversity of the ZINC database. It is also evident that FDA-approved drugs are concentrated in certain clusters, which promotes the development of molecules within these clusters. FIG. 9C also illustrates this space divided into 10 clusters, which can then be taken as a basis for sampling molecules from and enforcing diversity among them.

Using clustering as the basis for the selection of molecules, those molecules which are predicted to affect the activity of miR21 with the least amount of uncertainty are selected and virtually screened for dicer inhibition and cellular toxicity. Lastly, five molecules were chosen from each of the inference datasets (ZINC), belonging to different clusters, with few or no toxic activity predictions, and without dicer inhibition. Lastly, ten candidate molecules were selected, six from the Zinc dataset and four from the Asinex dataset. Ultimately, 8 final candidate molecules were selected for further experimentation due to the difficulty in purchasing two of Asinex's molecules. It was ensured that none of these molecules had been previously reported on the web and that they were entirely new.

### RNA Seq Analysis

In the first phase of evaluating the proposed candidate molecules with RiboStrike, the molecules were sorted according to their systematic effects on the expression of microRNA-21 target mRNAs based on gene set enrichment analysis. A total of eight candidate molecules were selected for validation, as illustrated in FIG. 13. The Z score was obtained using the miR21 knockout as a positive control for the miR21 ASO, which was compared to a negative control ASO. Among the chosen molecules from zinc, five candidates are selected with a confidence score of over 85%. Consequently, the model has a hit rate of 62.5%, higher than the in silico predicted precision score of 20.93% for the used model. Having a high hit rate is one of the main desired properties of a virtual screening model, which is why in this Example average precision score is used to give importance to the number of true positives and penalize false positives, resulting in less number of drug candidates being falsely predicted as hits.

### Reporter Assay

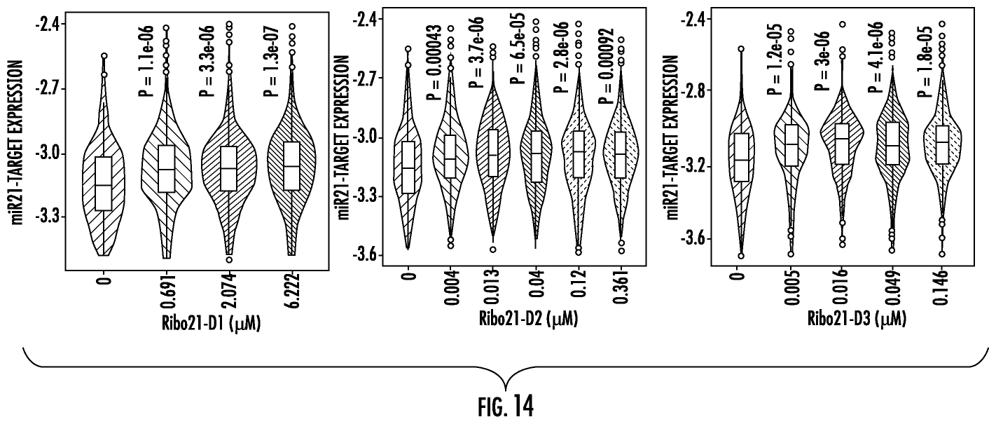

For the second validation round, a reporter assay was designed to assess the activity of five selected candidate hits. In FIG. 14, only candidates with promising results are shown, and the log ratio of GFP (Which carries miR21 targets in it) to mCherry is plotted. Among the five candidates validated from the last experiment, three were confirmed, giving the platform a hit rate of 37.5%.

### Mouse Experiment

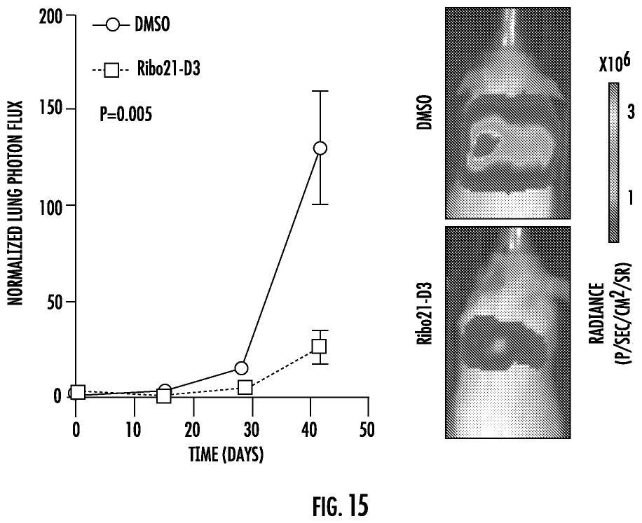

As the final phase of validation, MDA-MB-231tr breast cancer cells are treated for 48 hours with the most promising candidate molecule, MCULE-9082109585. Following the injection of the cells, the tumor changes have been monitored for a period of 40 days. The candidate molecule, as shown in FIG. 15, displayed the ability to decrease the growth pace of the tumor cells within the mouse in comparison to the control tumor.

### Language Modeling for Small Molecules

In recent years, language models have gained popularity in modeling text within the natural language processing field as well as other sequence-based data such as proteins and even small molecules within the biology and chemistry fields. These models have the capability to learn from unlabeled sequences solely by relying on a mechanism called “masked token prediction”, where certain words or “tokens” (e.g. amino acids, nucleotide bases, or atoms within a SMILES string) are omitted and the model is asked to predict the missing tokens. By doing so, the model can learn the grammar and syntax of the sequences, and with this knowledge, create meaningful representations for these sequences. ChemBERTa is a language model trained on 77 million SMILES strings from PubChem, which deploys masked token prediction and has a distinctive feature space to distinguish small molecules given their SMILES.

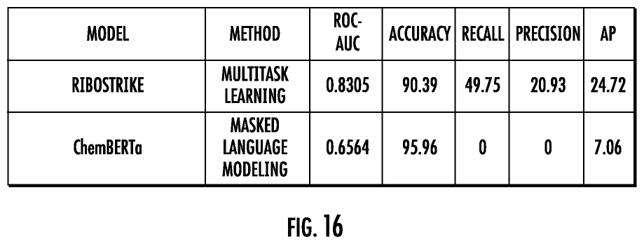

To assess the performance of the GCNN-based model described herein, it is compared to ChemBERTa. The pre-trained model was taken from HuggingFace repository and modified to be fit for a classification task by adding one fully connected layer to the end of the model. The weights of the base model were frozen during training and the model was trained for 2 epochs to predict the binary labels of the miR-21 task. The results are shown in the table of FIG. 16, which illustrates performance of the best model described herein compared to ChemBERTa on the test set of the miR-21 dataset.

As can be seen from FIG. 16, RiboStrike can empirically outperform ChemBERTa, with the help of multitask learning as well as task recommendation. While the comparison made here is for using ChemBERTa as a feature extractor as it is the prevalent approach, it is noteworthy to mention that the pipeline of RiboStrike can be model-agnostic and the principals of multitask learning and task recommendation can be extended to any model, even pre-trained language models such as ChemBERTa.

### Discussion

In light of microRNAs' dynamic structure and small size, small molecule discovery for the purpose of targeting microRNAs presents a number of challenges. It is also important to note that binding small molecules to their structure would not interfere with their function, which further complicates the situation. Considering the fact that many of their upstream players are unknown, structural approaches to drug discovery would be rendered ineffective. Considering this, systems and methods for discovering candidate molecular hits against microRNA activity before identifying its precise target are described herein. Accordingly, a virtual screening pipeline that uses deep learning to allow the selection of early hit candidates out of a large collection of diverse molecules is described. Multiple methods were implemented in order to ensure the practicality of the computational methods, resulting in the validation of multiple compounds in vitro and a single compound in vivo. The first step involved the use of deep learning models to learn from a large number of small molecule datasets since these models can learn patterns based on the input data and tailor their internal representations in accordance with the input data. By using multiple datasets and the labels corresponding to these datasets for training, this capability was amplified, enriching the models with the patterns found in these datasets. Additionally, a task recommendation technique was also introduced, which recommends how the datasets can be combined before training, in order to improve the model's performance after training. It has been observed that this technique has been successful empirically, and it has been hypothesized that by grouping tasks with similar predictions for training, the SGD process has a much easier time learning from the given data points and their corresponding gradient vectors when it comes to learning. The third methodology involved calculating uncertainty for all predictions made by the models, which enabled the ranking of molecules in spite of their binary nature. To finalize, the internal features of the model were used to represent molecules during inference, and clustering of these inner features allowed the selection of a diverse set of molecules for in vitro testing. In summary, the RiboStrike pipeline identified three potential hit candidates, demonstrating the advantage of using graph-based deep learning to identify hidden patterns of molecular hits against the activity of microRNAs without the need for sequence reading or structural information.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The invention claimed is:

1. A method for training a deep learning model configured for virtual screening of molecules for micro ribonucleic acid (miRNA) drug discovery, comprising:
creating a training dataset comprising a plurality of assay datasets, wherein the plurality of assay datasets comprise at least one miRNA assay dataset;
training the deep learning model to learn a plurality of tasks using the training dataset, wherein each of the plurality of assay datasets is associated with a respective task;
performing, using the deep learning model, inference for the respective task associated with the at least one miRNA assay dataset, wherein the deep learning model outputs a respective prediction for each of the plurality of tasks;
evaluating respective performance metrics associated with the respective predictions for each of the plurality of tasks;
selecting a set of the plurality of assay datasets for training based on the respective performance metrics; and
training the deep learning model to learn a set of the plurality of tasks using the set of the plurality of assay datasets, wherein the set of the plurality of assay datasets comprises the at least one miRNA assay dataset, and wherein the trained deep learning model is configured to predict molecules capable of affecting a target miRNA.

2. The method of claim 1, wherein the step of training the deep learning model to learn the plurality of tasks using the training dataset is performed in a multi-task manner.

3. The method of claim 1, wherein the step of evaluating respective performance metrics associated with the respective predictions for each of the plurality of tasks comprises calculating respective scores based on a comparison of the respective predictions for each of the plurality of tasks to ground truth labels for the respective task associated with the at least one miRNA assay dataset.

4. The method of claim 3, wherein the set of the plurality of assay datasets are associated with respective scores greater than a threshold.

5. The method of claim 1, wherein the target miRNA is miR-21.

6. The method of claim 1, wherein the trained deep learning model is configured to predict molecules that disrupt activities of the target miRNA.

7. The method of claim 6, wherein the trained deep learning model is configured to predict molecules that inhibit activity of the target miRNA or a protein upstream of the target miRNA.

8. The method of claim 1, wherein the deep learning model is a graph convolutional neural network (GCNN).

9. The method of claim 1, wherein the training dataset comprises a plurality of molecules expressed in a computer-readable format.

10. The method of claim 9, wherein the computer-readable format is simplified molecular input line entry system (SMILES) notation.

11. A method for screening of molecules for micro ribonucleic acid (miRNA) drug discovery, comprising:
providing a deep learning model trained according to claim 1;
inputting an inference dataset into the trained deep learning model;
predicting, using the trained deep learning model, a plurality of molecules capable of interacting with the target miRNA; and
performing in vitro testing on at least one of the plurality of molecules predicted as capable of affecting the target miRNA.

12. The method of claim 11, further comprising:
providing respective uncertainty scores for each of the plurality of molecules predicted as capable of interacting with the target miRNA; and
selecting the at least one of the plurality of molecules on which in vitro testing is performed based, at least in part, on the respective uncertainty scores.

13. The method of claim 11, further comprising:
clustering the plurality of molecules predicted as capable of interacting with the target miRNA into a plurality of clusters; and
selecting the at least one of the plurality of molecules on which in vitro testing is performed based, at least in part, on at least two of the plurality of clusters.

14. The method of claim 11, further comprising:
inputting the plurality of molecules predicted as capable of interacting with the target miRNA into a first machine learning model;
predicting, using the first machine learning model, respective toxicity metrics for the plurality of molecules predicted as capable of interacting with the target miRNA; and
selecting the at least one of the plurality of molecules on which in vitro testing is performed based, at least in part, on the respective toxicity metrics.

15. The method of claim 11, further comprising:
inputting the plurality of molecules predicted as capable of interacting with the target miRNA into a second machine learning model;
predicting, using the second machine learning model, respective dicer activity metrics for the plurality of molecules predicted as capable of interacting with the target miRNA; and
selecting the at least one of the plurality of molecules on which in vitro testing is performed based, at least in part, on the respective dicer activity metrics.

16. The method of claim 1, wherein the inference dataset comprises a plurality of molecules expressed in a computer-readable format.

17. The method of claim 16, wherein the computer-readable format is simplified molecular input line entry system (SMILES) notation.

18. (canceled)

19. (canceled)

20. (canceled)

21. A system for training a deep learning model configured for virtual screening of molecules for micro ribonucleic acid (miRNA) drug discovery, comprising:
at least one processor; and
at least one memory operably coupled to the at least one processor, the at least one memory having computer-executable instructions stored thereon that, when executed by the at least one processor, cause the at least one processor to:
receive a training dataset comprising a plurality of assay datasets, wherein the plurality of assay datasets comprise at least one miRNA assay dataset;
train the deep learning model to learn a plurality of tasks using the training dataset, wherein each of the plurality of assay datasets is associated with a respective task;
perform, using the deep learning model, inference for the respective task associated with the at least one miRNA assay dataset, wherein the deep learning model outputs a respective prediction for each of the plurality of tasks;
evaluate respective performance metrics associated with the respective predictions for each of the plurality of tasks;
select a set of the plurality of assay datasets for training based on the respective performance metrics; and
train the deep learning model to learn a set of the plurality of tasks using the set of the plurality of assay datasets, wherein the set of the plurality of assay datasets comprises the at least one miRNA assay dataset, and wherein the trained deep learning model is configured to predict molecules capable of affecting a target miRNA.

22. The system of claim 21, wherein the step of training the deep learning model to learn the plurality of tasks using the training dataset is performed in a multi-task manner.

23. The system of claim 21, wherein the step of evaluating respective performance metrics associated with the respective predictions for each of the plurality of tasks comprises calculating respective scores based on a comparison of the respective predictions for each of the plurality of tasks to ground truth labels for the respective task associated with the at least one miRNA assay dataset.

24. The system of claim 23, wherein the set of the plurality of assay datasets are associated with respective scores greater than a threshold.

25. The system of claim 21, wherein the target miRNA is miR-21.

26. The system of claim 21, wherein the trained deep learning model is configured to predict molecules that disrupt activities of the target miRNA.

27. The system of claim 26, wherein the trained deep learning model is configured to predict molecules that inhibit activity of the target miRNA or a protein upstream of the target miRNA.

28. The system of claim 21, wherein the deep learning model is a graph convolutional neural network (GCNN).

29. The system of claim 21, wherein the training dataset comprises a plurality of molecules expressed in a computer-readable format.

30. The system of claim 29, wherein the computer-readable format is simplified molecular input line entry system (SMILES) notation.

31-35. (canceled)


SEQUENCE LISTING (1 sequence)

SEQ ID NO: 1 - DNA, 22 bp, Homo sapiens
  source 1..22: mol_type = other DNA
tcaacatcagtctgataagcta